(19) United States
(12) Patent Application Publication
(10) Pub. No.: US 2004/0049164 A1
(43) Pub. Date: Mar. 11, 2004

(54) System including a tapered entry into an injection needle

(75) Inventors: Charles J. Patrick, Milwaukee, WI (US); Dean A. Erickson, Greenfield, WI (US)

Correspondence Address: FOLEY & LARDNER, 321 NORTH CLARK STREET, SUITE 2800, CHICAGO, IL, 60610-4764, US

(73) Assignee: BioForm, Inc.

(21) Appl. No.: 10/235,210

(22) Filed: Sep. 6, 2002

(51) Int. Cl. A61M 5/32

(52) U.S. Cl. 604/272; 604/240; 604/164.01

(57) ABSTRACT

An injection system comprising a tube having an inner diameter and an injection needle including a leading edge at one end thereof. A tapered surface is formed between a portion of the tube and the leading edge of the needle. The tapered surface provides a smooth transition surface from the tube to the needle, preventing the needle assembly from plugging when suspended particle materials are used in the injection system and allowing for easy and thorough cleaning of the needle for reuse and/or sterilization.

### FIELD OF THE INVENTION

The present invention is directed generally to medical delivery systems. More particularly, the present invention is related to medical delivery systems that provide a smooth transition surface along which injectable materials can flow through the inside of a needle.

### BACKGROUND OF THE INVENTION

Medical delivery systems, such as those comprising syringe and needle assemblies, are commonly used for the purpose of injecting fluids into or withdrawing fluids from a body. These fluids may include medicine, blood or other types of biological materials.

Although conventional medical delivery systems function in an adequate manner for a number of purposes, such systems currently include a number of limitations. For example, when injecting particles that are suspended in a gel carrier, the particles may lodge on the needle's leading edge or wall. If particles continue to collect in this location, the particles may eventually plug the passageway. If the passageway becomes plugged, the injection material and the particles contained therein are prevented from traveling down the needle and being placed into the body tissue.

Additionally, in conventional injection systems there is often no seal of the flexible tubing to the needle at the needle's leading edge. For most flexible injection needles, the needle is bonded by adhesive or formed via molding into the flexible injection tubing. This allows for a varying or constant thickness gap to form at the needle's leading edge between the needle and the tubing; into which material can flow. The gap is closed off at the point the needle is actually sealed or bonded to the tubing, forming a single sided entrance/exit into the gap's containment (volume) created. In the case of materials with suspended particles, such as the tissue augmentation material described in U.S. Pat. No. 5,922,025 issued to Hubbard, a narrow gap may allow for the suspended particle carrier to flow into the gap but not the suspended particles. If there is enough carrier separation, the suspended particles will stop flowing, the needle will plug and the injection material will be prevented from being placed into the tissue. Also, this gap around the leading edge of the needle and its associated volume makes it impossible to thoroughly clean the needle because of the extreme difficulty in accessing the narrow gap.

For most injection materials, such as liquid drugs, this gap and needle edge is not a major issue because the material flows much easier and does not contain suspended particles which can separate out. For injection materials that contain suspended particles, however, the above issues are much more of a concern.

Prior art in FIGS. 1-3 show three conventional tube/needle connection designs. In each design, a needle 20 is sized to fit within a tube 22. In prior art FIG. 1, an adhesive 28 is used to bond the needle 20 to the tube 22. In prior art FIGS. 2 and 3, the tube 22 is formed such that a snug fit exists between the tube 22 and the needle 20. In each of these cases, however, a leading edge 24 of the needle creates a “ledge” with the inner wall 26 of the tube 22, resulting in a location where material is capable of collecting.

### SUMMARY OF THE INVENTION

It is therefore an object of the invention to provide an improved delivery system that provides a smooth transition from the system's tubing to the needle.

It is another object of the invention to provide an improved delivery system that seals the needle's leading edge to the associated tubing.

It is still another object of the invention to provide an improved delivery system where the full inside diameter of the needle can be cleaned and/or sterilized so that the needle can be reused.

It is another object of the invention to provide an improved delivery system that permits a stylet to be easily inserted through the junction of the needle and the tubing.

In accordance with the above objects, a medical injection system comprises a tube having an inner diameter, and an injection needle including a leading edge at one end thereof. A tapered surface is formed between a portion of the tube and the leading edge of the needle. The tapered surface provides a smooth transition surface from the tube to the needle, preventing the needle from plugging when suspended particle materials are used in the injection system and/or allows for easy cleaning of the inner diameter of the needle system. A variety of methods and arrangement can be used to form the tapered surface between the tube and the needle.

### BRIEF DESCRIPTION OF THE DRAWINGS

The foregoing advantages and features of the invention will become apparent upon reference to the following detailed description and the accompanying drawings, of which:

FIG. 1 is a sectional side view of a first type of prior art tube/needle assembly;

FIG. 2 is a sectional side view of a second type of prior art tube/needle assembly;

FIG. 3 is a sectional side view of a third type of prior art tube/needle assembly;

FIG. 4 is a sectional side view of a tube/needle assembly according to a first embodiment of the invention;

FIG. 5 is a sectional side view of a tube/needle assembly according to a second embodiment of the invention;

FIG. 6 is a sectional side view of a tube/needle assembly according to a third embodiment of the invention;

FIG. 7 is a sectional side view of a tube/needle assembly according to a fourth embodiment of the invention;

FIG. 8 is a sectional side view of a tube/needle assembly according to a fifth embodiment of the invention;;

FIG. 9 is a sectional side view of a tube/needle assembly according to a sixth embodiment of the invention;

FIG. 10 is a side view of a medical delivery system according to one embodiment of the invention with the syringe body separated from the needle assembly and

FIG. 11 is a side view of the medical delivery system of FIG. 10 in the fully assembled position.

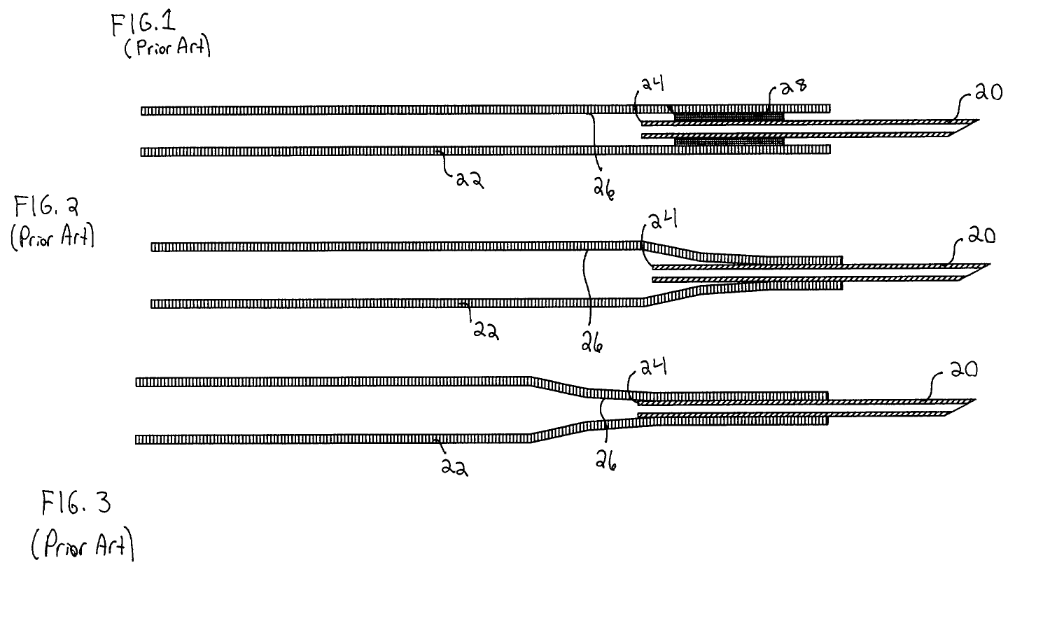

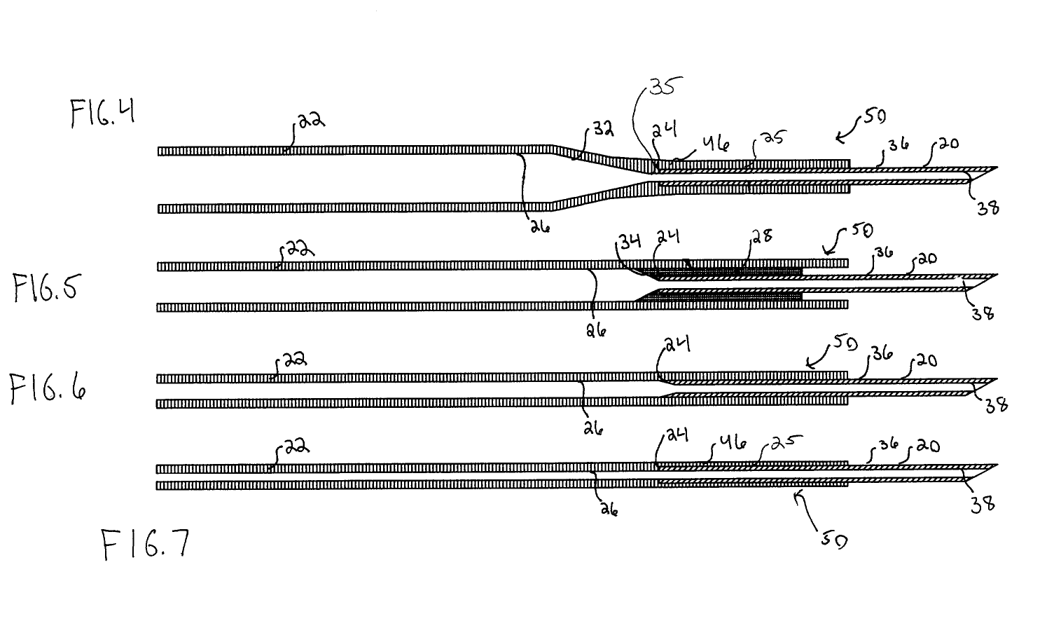

### DETAILED DESCRIPTION OF THE INVENTION

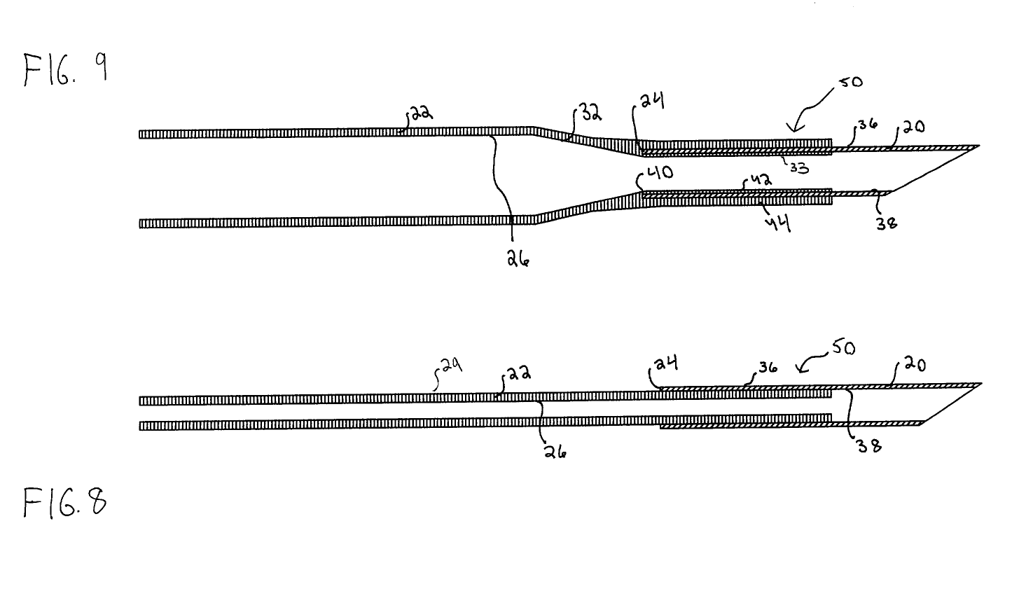

A tube/needle assembly constructed in accordance with the present invention is shown generally at 50 in FIGS. 4-9. The tube/needle assembly 50 comprises a tube 22 coupled to a needle 20. The tube 22 includes an inner surface 26, while the needle 20 includes a leading edge 24 at one end thereof (see FIG.4), a diameter of the outer surface 36 and a diameter of an inner surface 38. Depending upon the particular requirements of the application, the tube 22 could be flexible, semi-flexible or rigid. Stainless steel is one type of material which could be used to form a rigid tube 22. In each of the embodiments shown in FIGS. 4-9, the smooth transition is formed between the diameter of the inner surface 26 of the tube 22 and the diameter of the inner surface 38 of the needle 20.

A variety of mechanisms and structures can be used for forming a smooth transition between the diameter of the inner surface 26 of the tube 22 and the diameter of the inner surface 38 of the needle 20. In FIG. 4, the tube 22 includes a transition portion 32 which results in a secondary inner surface 35 which diameter is smaller than the diameter of the inner surface 26 of the remainder of the tube 22. The portion of the tube 22 that includes the secondary inner surface 35 also includes an indentation 25. The indentation 25 is sized such that the needle 20 can fit therein with its leading edge 24 abutting against a portion of the tube 22. As a result, the diameter of the inner surface 38 of the needle 20 is substantially identical in size to the secondary inner surface 35 of the tube 22. By having a smooth transition between the tube 22 and the needle 20, the likelihood of materials collecting near the leading edge 24 of the needle 20 decreases substantially.

FIG. 7 is similar to FIG. 4 in that a portion of the needle 20 fits inside an indentation 25 of the tube 22. In this embodiment, however, there is no transition portion 32 within the tube 22, resulting in a single diameter of the inner surface 26 up until the indentation 25. Nevertheless, the leading edge 24 of the needle 20 still substantially abuts against a portion of the tube 22, resulting in a smooth transition between the diameter of the inner surface 26 of the tube and the diameter of the inner surface 38 of the needle 20. In either of the embodiments shown in FIGS. 4 or 7, the indentation 25 can be machined, formed or molded into the tube 22 before the needle 20 is inserted. The needle 20 may also be bonded or molded to the inside of the tube 22 if so desired.

FIG. 5 shows another embodiment of the invention. In this embodiment, the diameter of the outer surface 36 of the needle 20 is substantially smaller than the diameter of the inner surface 26 of the tube 22. An adhesive, such as epoxy, is used to bond the needle 20 to the inside of the tube 22. The adhesive 28 is applied in the tube needle assembly 50 in a manner such where the tapered surface 34 is formed up to the leading edge 24 of the needle 20. As a result, a smooth transition is formed from the diameter of the inner surface 26 of the tube across the tapered surface 34 of the adhesive 28 to the diameter of the inner surface 38 of the needle 20.

Another embodiment of the invention is shown in FIG. 6. In this particular embodiment, the diameter of the outer surface 36 of the needle 20 is substantially identical to the diameter of the inner surface 26 of the tube 22. The leading edge 24 of the needle 20 is tapered to form a smooth transition between the inner surface 26 of the tube 22 and the inner surface 38 of the needle 20.

FIG. 8 shows yet another embodiment of the invention. In this embodiment, the diameter of an outer surface 29 of the tube 22 is substantially identical to the diameter of the inner surface 38 of the needle 20. In this arrangement, the tube 22 is capable of being inserted directly into the inside of the needle 20. Because the leading edge 24 of the needle 20 is located on the outside of the tube 22, the leading edge 24 will not promote a collection or buildup of material inside the tube 22.

FIG. 9 shows still another embodiment of the invention. In the embodiment shown in FIG. 9, the tube 22 includes a transition portion 32 that leads to a reduced inner surface 33, which defines a reduced portion 44 of the tube 22. The reduced portion 44 of the tube 22 also includes a slot 40 formed therein. The slot 40 is sized to accept the leading edge 24 of the needle 20. With the leading edge 24 and the needle 20 contained within the tube 22, a smooth transition surface is formed which prevents material from collecting near the leading edge 24 of the needle 20.

As described above, the process used to manufacture the smooth transition surface from the inner surface 26 of the tube 22 to the inner surface 38 of the needle 20 can include a variety of forms. These include:

1) using an adhesive or other similar material to form a taper, which covers the needle's leading edge; 2) machining, forming, or molding a taper and ledge in the end of the tubing, and then inserting and bonding the needle into the formed pocket; 3) directly molding the needle into the end of the tube 4) maintaining the inside tubing diameter the same as the inside needle diameter and providing a smooth transition from the tubing to the needle; 5) maintaining the tubing with an inside diameter approximately the same as the outside diameter of the needle and then tapering the inside of the needle to provide the smooth transition; 6) providing the tubing with an outside diameter approximately the same as the inside diameter of the needle and 7) various combinations of 1 through 6.

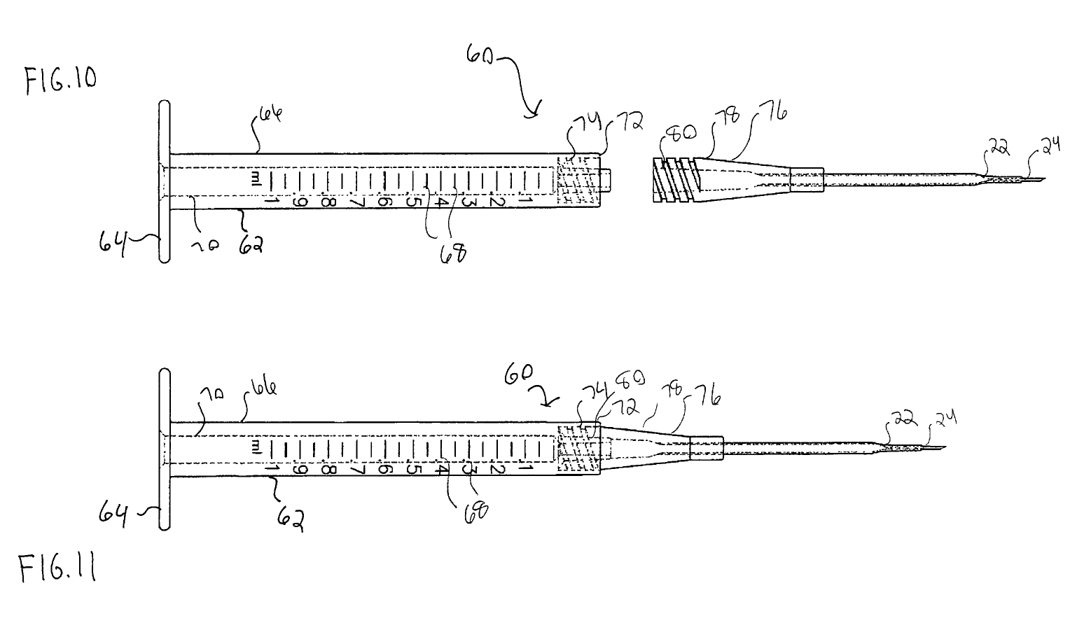

A medical delivery or extraction system 60 constructed in accordance with the present invention is shown in FIGS. 10 and 11. The delivery or extraction system 60 comprises a syringe 62 and a needle assembly 76. A syringe body 66 may accept a plunger (not shown) that is used to force fluid into or out of the syringe 62. The syringe body 66 also includes a plurality of volume indicia 68 for measuring the amount of fluid inside the syringe 62 at any given moment. The syringe 62 includes an inner body 70 (shown in phantom in FIGS. 10 and 11) on the inside of the syringe 62. The inner body 70 comprises a passageway through which the fluid primarily flows. The inner body 70 terminates at a connection portion 72. In the area around the connection portion 72, there are a plurality of threads 74 (shown in phantom) formed on the inside of the syringe body 66. The needle assembly 76 comprises the needle 24 coupled to the tubing 22 which runs to a luer connection 78. A stylet (not shown) can also be inserted into the needle assembly 76. The luer connection 78 includes a hub 80 (shown in phantom) formed on the inside thereof. The hub 80 is used to mate the needle assembly 76 with the syringe 62.

The present invention can be used in conjunction in a variety of environments ranging from sterile hospital suites to non-sterile offices, primarily at ambient conditions. Additionally, the individual components could be color coded to an industry standard, indicating a particular feature, such as needle gauge of the product. The plastic tubing used can be manufactured from readily available extruded tubing known to those in the art. The needle 20 and associated components can be produced by current extrusion and fabrication methods such as grinding, drilling, cutting, milling, and polishing. The assembly of the tube/needle assembly 50 can be completed with standard assembly, forming, bonding, printing, and molding operations. The system can be packaged and sterilized using currently available methods.

It should be understood that the above description of the invention and specific examples and embodiments, while indicating the preferred embodiments of the present invention, are given by demonstration and not limitation. For example, the tube/needle assembly 50 could be manufactured with any needle or flexible, semi flexible or rigid tubing size in which the suspended particles can flow through. An injection system according to the present invention may also include an adjustable outer sheath that covers the needle 20 that tapers and/or blends into the needle's tip during part of the injection procedure. Many changes and modifications within the scope of the present invention may therefore be made without departing from the spirit thereof and the present invention includes all such changes and modifications.

What is claimed is:

1. An injection system, comprising:
a tube having an inner diameter;
an injection needle including a leading edge at one end thereof, and
means for providing a tapered surface between a portion of the tube and the leading edge of the injection needle.

2. The injection system of claim 1, wherein the injection needle extends partially within the tube.

3. The injection system of claim 2, wherein the means for providing a tapered surface comprises a molded end portion on the tube, the molded end portion forming a smooth transition portion with the leading edge of the injection needle.

4. The injection system of claim 2, wherein the means for providing a tapered surface comprises an adhesive bonding the inside of the tube to the outside of the injection needle, the adhesive forming a tapered surface between the leading edge of the needle and the tube.

5. The injection system of claim 2, wherein the means for providing a tapered surface comprises a transition portion on the leading edge of the injection needle.

6. The injection system of claim 2, wherein the means for providing a tapered surface comprises an indented region on the inside of the tube, the indented region having a diameter such that the outer diameter of the needle fits and the inner diameter of the injection needle is substantially identical to the inner diameter of the flexible tube at the start of the indented region.

7. The injection system of claim 2, wherein the means for providing a tapered surface comprise either or both:
a slot formed between the inner diameter of the flexible tube and an outer diameter of the tube for accepting the injection needle;
a transition surface along the inner diameter of the tube, the inner diameter decreasing as the transition surface approaches the injection needle.

8. The injection system of claim 2, wherein the tube comprises a secondary transition portion, the tube decreasing in diameter as the transition portion approaches the leading edge of the injection needle.

9. The injection system of claim 2, wherein the tube comprises a substantially constant inner diameter up to the leading edge of the injection needle.

10. The injection system of claim 1, further comprising a stylet for insertion inside the injection needle.

11. The injection system of claim 1, wherein the tube extends partially within the injection needle.

12. The injection system of claim 1, wherein the tube is flexible.

13. The injection system of claim 1, wherein the tube is semi-flexible.

14. The injection system of claim 1, wherein the tube is rigid.

15. An injection assembly, comprising:
a syringe;
a tube having an inner diameter and operatively connected to the syringe;
a needle including a leading edge at one end thereof, and
a tapered surface formed between a portion of the tube and the leading edge of the needle.

16. The injection assembly of claim 15, wherein the tapered surface is formed from a molded end portion on the tube, the molded end portion forming a smooth transition portion with the leading edge of the needle.

17. The injection assembly of claim 15, wherein the tapered surface is formed from an adhesive bonding the inside of the tube to the outside of the needle.

18. The injection assembly of claim 15, wherein the tapered surface is formed along the leading edge of the needle.

19. The injection assembly of claim 15, wherein the tapered surface comprises an indented region on the inside of the tube, the indented region having a depth such that the inner diameter of the needle is substantially identical to the inner diameter of the tube at the indented region.

20. The injection assembly of claim 15, further comprising a stylet for insertion inside the needle.

21. The injection assembly of claim 15, wherein the tapered surface comprises a secondary transition portion that decreases in diameter as the transition portion approaches the leading edge of the injection needle.

22. The injection assembly of claim 15, wherein the tube comprises a substantially constant inner diameter up to the leading edge of the injection needle.

23. The injection assembly of claim 15, wherein the tube is flexible.

24. The injection assembly of claim 15, wherein the tube is semi-flexible.

25. The injection assembly of claim 15, wherein the tube is rigid.

26. The injection assembly of claim 25, wherein the tube is formed from stainless steel.

27. A medical delivery system, comprising:
a syringe;
a luer assembly operatively connected to the syringe;
a connection tube having an inner diameter and operatively connected to the luer assembly;
a needle including a leading edge at one end thereof, and
means for providing a tapered surface between a portion of the connection tube and the leading edge of the needle.
